(19) United States
(12) Patent Application Publication
(10) Pub. No.: US 2023/0363743 A1
(43) Pub. Date: Nov. 16, 2023

(54) SPECIMEN COLLECTION DEVICE

(71) Applicants: Mayo Foundation for Medical Education and Research, Rochester, MN (US); Safe Health Systems, Inc., Los Angeles, CA (US)

(72) Inventors: Ken Mayer, Los Angeles, CA (US); Thomas E. Grys, Scotsdale, AZ (US)

(21) Appl. No.: 18/013,673

(22) PCT Filed: Jun. 17, 2021
(86) PCT No.: PCT/US2021/037840

§ 371 Date: Dec. 29, 2022

Related U.S. Application Data
(60) Provisional application No. 63/120,706, filed on Dec. 2, 2020.
(60) Provisional application No. 63/047,792, filed on Jul. 2, 2020.

(51) Int. Cl. A61B 10/00

(52) U.S. Cl. CPC A61B 10/0045

(57) ABSTRACT

This specification discloses devices and apparatus for collecting a material specimen, including a sheath; and an inner assembly. The inner assembly can include a handling element; one or more fluid chamber elements; and a swab element. The handling element, the one or more fluid chamber elements, and the swab element are connected and arranged along a first axis; and the inner assembly is operable to slide within the sheath between (i) a first position in which the swab element is in a non-retracted position outside of the sheath while at least a portion of the inner assembly remains within the sheath, and (ii) a second position in which the swab element is in a retracted position within the sheath.

### CROSS-REFERENCE TO RELATED APPLICATIONS

The application claims the benefit of priority to U.S. provisional application Ser. No. 63/047,792, filed Jul. 2, 2020, and U.S. provisional application Ser. No. 63/120,706, filed Dec. 2, 2020. The entire contents of these applications are considered part of the present disclosure and are incorporated by reference in their entireties.

### FIELD OF THE DISCLOSURE

The disclosure relates to methods and materials for the collection of biological specimens (e.g., for testing), including techniques that permit collection of more than one type of biological specimen using the same collection device for facile delivery to a test kit.

### BACKGROUND

Systems and technologies that facilitate self-collection of biological specimens and other material specimens are available in consumer markets. These specimens can be used for a range of testing methods and can utilize a number of specimen collection modalities. Collected specimens are sometimes mixed with a reagent solution for a lateral flow immunoassay (“LFIA”) test in a separate container, and subsequently applied to an application location on a test strip or cassette. As the specimen migrates to and reacts with reagents present in one or more analytical areas, a test result can be indicated to the user according to instructions provided by the test supplier.

### SUMMARY

This disclosure relates to a collection device that can allow for the collection of multiple specimen types, and methods for use of the device.

Disclosed herein is a collection device for the collection of multiple specimen types and potential use in conjunction with multiple lateral flow immunoassay (LFIA) assays and similar devices. Some implementations of the device may, in certain cases, achieve one or more advantages such as being more user friendly, less prone to human error, capable of improving test validity, and capable of improving user safety by protecting a LFIA-specific reagent solution within the device and applied to the specimen by the user after collection. The device can be used in a range of specimen collection modalities including oral or nasopharyngeal swabs, serological specimen absorption, or fecal swabs.

In a first aspect, a device is provided for collecting a material specimen, including a sheath; and an inner assembly. The inner assembly including a handling element; one or more fluid chamber elements; and a swab element. The handling element, the one or more fluid chamber elements, and the swab element are connected and arranged along a first axis; and the inner assembly is operable to slide within the sheath between (i) a first position in which the swab element is in a non-retracted position outside of the sheath while at least a portion of the inner assembly remains within the sheath, and (ii) a second position in which the swab element is in a retracted position within the sheath.

Embodiments of the device for collecting a material specimen can include one or more of the following features. The sheath can include a second axis, and the first axis of the inner assembly can be configured to slide coaxially along the second axis of the sheath.

The handling element can be located at a proximal end of the inner assembly, the swab element can be located at a distal end of the inner assembly, and the one or more fluid chamber elements can be located intermediately between the proximal end of the inner assembly and the distal end of the inner assembly. The inner assembly further can include a shaft that extends along the first axis between the one or more fluid chamber elements and the swab element.

The handling element can include a compressible bulb configured to expel a pressurized gas to the one or more fluid chamber elements when the bulb can be compressed, thereby causing a fluid within the one or more fluid chamber elements to be expressed toward the swab element. The inner assembly can be configured such that expulsion of the pressurized gas from the bulb when compressed causes a seal in the one or more fluid chamber elements to be broken to permit flow of the fluid.

The material specimen can include saliva, urine, blood, or serum of a person.

The device can be configured to collect material specimens of multiple types.

Actuation of the inner assembly can break a seal in the one or more fluid chamber elements to permit a flow of a fluid from the one or more fluid chamber elements.

At least one of the handling element or the swab element can be a modular component that can be removably connected to the inner assembly.

In a second aspect, a device is provided for collecting a material specimen, including an assembly. The assembly includes a handling element; one or more fluid chamber elements; and a swab element. The handling element, the one or more fluid chamber elements, and the swab element are connected and arranged along a first axis. Other advantages will be apparent from the description, the drawings, and the claims.

### BRIEF DESCRIPTION OF THE DRAWINGS

FIG. 1A is a schematic diagram depicting the collection device prior to actuation by a user.

FIG. 1B is a schematic diagram depicting the collection device after actuation by a user, and a punctured solution chamber.

FIG. 1C is a schematic diagram depicting the collection device after actuation by a user, the solution chamber being emptied through the shaft and into the swab.

FIG. 2A is a schematic diagram of an exemplary collection device with a removable sheath.

FIG. 2B is a schematic diagram showing the swab and shaft removed from an exemplary collection device with a removable sheath.

FIG. 2C is a schematic diagram showing an exemplary collection device with optional elements affixed to the sheath and handling element.

FIG. 3A is a schematic diagram of an exemplary collection device with a removable sheath and a tip element.

FIG. 3B is a schematic diagram showing the swab and shaft removed from an exemplary collection device with a removable sheath and tip element.

FIG. 4 is a schematic diagram of an exemplary collection device with an integrated construction.

FIG. 5A is a schematic diagram of an exemplary collection device with an elemental construction.

FIG. 5B is a schematic diagram of an exemplary handling area element with two solution chambers and a screw-type attachment.

FIG. 5C is a schematic diagram of an exemplary handling area element with one solution chamber and a snap-type attachment.

FIG. 5D is a schematic diagram of exemplary interchangeable swab and tip elements.

FIG. 6 is a schematic diagram of an exemplary collection device with an actuator and a removable sheath.

FIG. 7A is a depiction of a first exemplary swab element.

FIG. 7B is a depiction of a second exemplary swab element.

FIG. 7C is a depiction of a third exemplary swab element.

FIG. 8A is a schematic diagram of an exemplary collection device with an actuator and tip element.

FIG. 8B is a schematic diagram of an exemplary collection device with the actuator showing a swab extending from a sheath.

FIG. 9A is a cross sectional view of an exemplary collection device with the actuator and the swab retracted into the sheath.

FIG. 9B is a cross sectional view of an exemplary collection device having the swab retracted into the sheath when the actuator is activated.

FIG. 10A is a cross sectional view of an exemplary collection device having the swab retracted into the sheath when the actuator is activated.

FIG. 10B is a cross sectional view of an exemplary collection device having a cap sealing the swab inside the sheath.

FIG. 10C depicts an exemplary rectangular cap that attaches to the sheath of an exemplary collection device.

FIG. 10D depicts an exemplary pointed cap that attaches to the sheath of an exemplary collection device.

FIG. 11A depicts an exemplary collection device having the actuator and the tip element.

FIG. 11B depicts another view of an exemplary collection device having the actuator and the tip element.

FIG. 11C depicts a side view of an exemplary collection device having the actuator and the tip element.

FIG. 11D is a cross sectional view of an exemplary collection device having the actuator and the tip element when the tip element is retracted inside the sheath.

FIGS. 12A-D respectively depict a front view, first side view, second side view, and cross-sectional view of an exemplary collection device.

FIG. 13 is a perspective view of an exemplary collection device.

In the figures, like symbols indicate like elements.

### DETAILED DESCRIPTION

This document describes a collection device configured to facilitate the collection of multiple specimen types.

A collected specimen may need to be processed with one or more additional solutions or reagents when tested with an LFIA testing device.

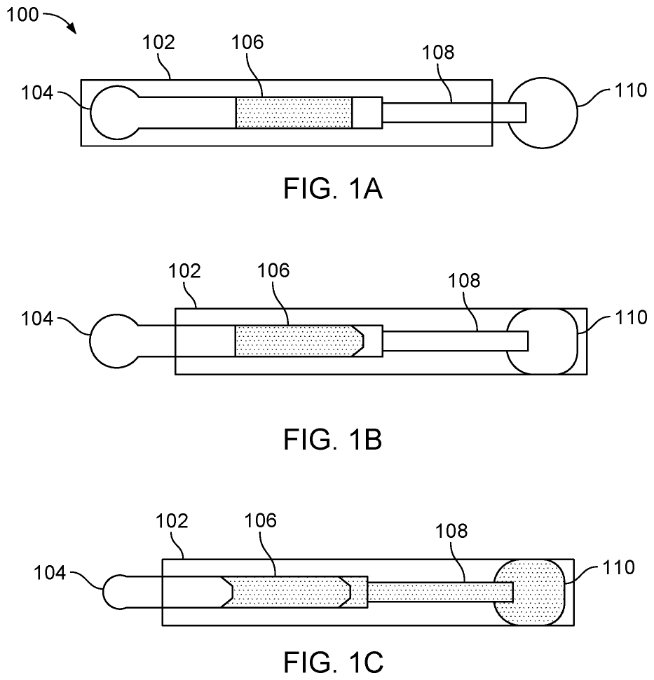

FIGS. 1A-1C depict an embodiment in which an enclosing shaft can be actuated by a user to perform the specimen collection. FIG. 1A depicts a collection device 100 which can be used by a user for the collection of a specimen for testing in a testing device, e.g., an LFIA testing device. The collection device 100 can include a sheath 102 which the user controls with a hand. The sheath 102 can define a hollow shaft that houses interior components of the collection device 100.

The interior components of the collection device 100 can include a handling area 104, a sealed solution chamber 106, and a shaft 108. In some embodiments, the handling area 104 is composed of a material capable of being deformed (e.g., plastic). In some embodiments, the handling area 104 is compressible (e.g., a bulb). The interior volume of the handling area 104 can be filled with a gas. In some embodiments, the gas can be pressurized. In use, the handling area 104 is positioned proximal to the user within the sheath 102 and is affixed to the proximal end of the sealed solution chamber 106.

The solution chamber 106 defines a chamber within the collection device 100 containing a volume of solution for processing the specimen prior to LFIA testing (e.g. an ampule, bladder, or pack). In general, the solution chamber 106 can be constructed of a material capable of being destructively deformed (e.g., glass, or plastic). For example, the solution can include, but is not limited to, a buffer, or a reagent suspension. Non-limiting examples of volumes the solution chamber 106 can contain include, but are not limited to, about 0.1 mL to about 3 mL (e.g., about 0.1 mL to about 3 mL, about 0.5 mL to about 3 mL, about 1 mL to about 3 mL, about 2 mL to about 3 mL, about 0.1 mL to about 2 mL, about 0.1 mL to about 1 mL, or about 0.1 mL to about 0.5 mL). In some embodiments, the solution chamber 106 contains one chamber. In some embodiments, the solution chamber 106 includes multiple chambers (e.g., two or more, three or more, four or more, etc.), each chamber containing a distinct solution.

The distal end of the solution chamber 106 can be affixed to a shaft 108. The sheath 102 can be made to fully enclose the handling area 104 and the sealed solution chamber 106, and to partially enclose the shaft 108. This configuration can allow the handling area 104 and sealed solution chamber 106 to be unaffected by user handling prior to the collection of the specimen.

In some embodiments, the shaft 108 is composed of a non-absorbent material that ensures no molecular shedding occurs as the solution chamber 106 solution is released (e.g., plastic, metal, or glass). The shaft 108 provides a hollow interior to allow the solution chamber 106 solution to flow through it from one end to the other. The length of the shaft 108 can be sized so as to facilitate convenient handling by the user. Non-limiting examples of the length of the shaft 108 can be about 2 cm to about 10 cm (e.g., about 2 cm to about 10 cm, about 4 cm to about 10 cm, about 6 cm to about 10 cm, about 8 cm to about 10 cm, about 2 cm to about 8 cm, about 2 cm to about 6 cm, or about 2 cm to about 4 cm).

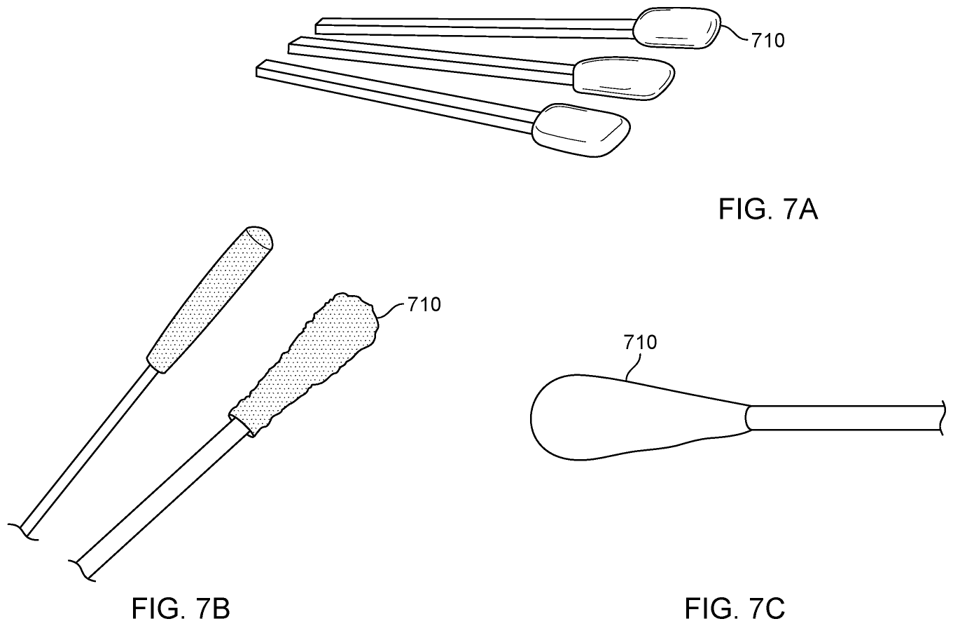

A swab 110 can be affixed to the distal end of the shaft 108 such that the swab 110 is left unenclosed by the sheath 102. The swab 110 can be composed of a natural or artificial material formed in a shape conducive to specimen collection. In some embodiments, the swab 110 can be formed through spinning, weaving, or flocking of the material. For example, the swab 110 material can include, but is not limited to, absorptive materials such as cotton, rayon, Dacron, polyester, or polymer foam. While the exemplary swab 110 in FIG. 1A is depicted as spherical, additional non-limiting examples of the shape of the swab 110 can include cylindrical, or hemispherical. Further exemplary swab 110 formations are depicted in FIG. 7.

The user can handle the sheath 102 in a manner to direct the swab 110 into contact with a specimen. The specimen can be a liquid, solid, gel, or semi-solid specimen collected from a subject, e.g., a human or other mammal or animal of interest. In some embodiments, the subject can be the user. Non-limiting examples of the specimen can include blood, urine, saliva, feces, or mucus.

The user can contact the swab 110 to the specimen (or tissue of the subject that contains the specimen) for a duration of time such that at least a portion of the specimen becomes affixed to the swab 110. In one non-limiting example, the swab 110 can be contacted to a liquid specimen (e.g., blood) for a duration that permits a portion of the liquid specimen to be absorbed by the swab 110. In general, the swab 110 can be contacted to the specimen at least one time (e.g., at least one time, at least two times, at least three times, at least four times, at least five times).

The sheath 102 is configured to actuate by sliding longitudinally along the length of the collection device 100 coaxially with the interior components. The user can actuate the collection device 100 to fully enclose the swab 110. That is, a user may pull the proximal end of the interior assembly (e.g., 104, 106, 108, and 110) by gripping the handling area 104 to slide the interior assembly toward the proximal end to cause the swab 110 to retract within the sheath 102. By pushing the interior assembly in the opposite direction, the retracted swab 110 can be removed from the sheath 102 to a non-retracted position. The sheath 102 can be of a length such that enclosing the swab 110 partially or fully exposes the handling area 104. FIG. 1B depicts the collection device 100 in an actuated position with an enclosed swab 110 retracted, an exposed handling area 104, and an unsealed solution chamber 106.

In some embodiments, actuating the collection device 100 can allow the user to destructively unseal the solution chamber 106. Non-limiting examples of methods of destructive unsealing can include piercing, perforating, cutting, bursting, or segmenting. For example, the sheath 102 can be configured such that it remains rigid (e.g., substantially not bendable or compressible under typical forces) while the collection device 100 is in the non-actuated position. In such embodiments, upon actuation, the user can further bend or compress the sheath 102 to destructively unseal the solution chamber 106.

In some embodiments, actuating the collection device 100 can cause the solution chamber 106 to be destructively unsealed without additional user interaction. In embodiments with more than one compartment within the sealed solution chamber 106, user actuation of the collection device 100 can unseal the compartments sequentially or concurrently. Further, user actuation can mix the contents of the one or more compartments.

With the collection device 100 in the actuated position, the exposed handling area 104 is accessible to and can be activated by the user. FIG. 1C depicts an example in which the handling area 104 is activated by the user through compression. For example, the user can use fingers to compress the handling area 104 and reduce the interior volume. In some embodiments, the increased pressure resulting from compression of the handling area 104 is sufficient to break a seal at the proximal end of the solution chamber 106. The unsealing of the proximal end of the solution chamber 106 can allow the increased pressure from handling area 104 compression to expel the solution from the solution chamber 106 into the shaft 108. In some embodiments, gravity can be allowed to express the solution into the shaft.

Referring further to FIG. 1C, the solution can be allowed to flow through the cavity and distal aperture of the shaft 108. The solution can then further flow into the swab 110 containing the specimen.

In general, the solution can be allowed to contact the specimen present on the swab 110 for a duration of time. A non-limiting range for the duration of time can be from about 1 s to about 1 hr (e.g., about 1 s to about 1 hr, about 10 s to about 1 hr, about 1 min to about 1 hr, about 10 min to about 1 hr, about 30 min to about 1 hr, about 1 s to about 30 min, about 1 s to about 10 min, about 1 s to about 1 min, about 1 s to about 10 s).

Following the contact time duration, the user can further use the handling area 104 to express the solution containing the specimen out of the actuated collection device 100. For example, the user can direct the expressed solution onto an LFIA or similar testing device for processing.

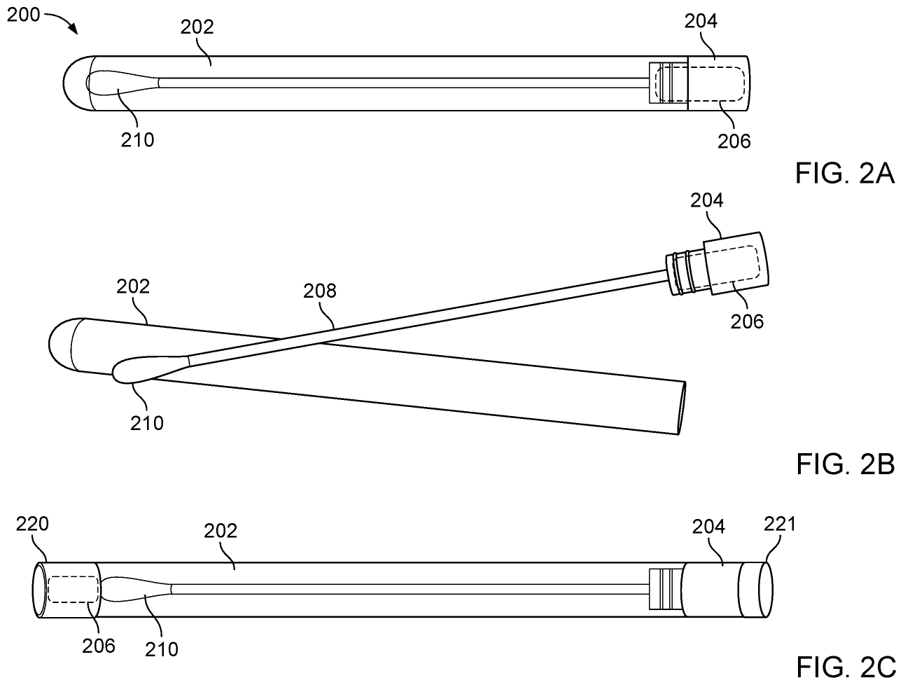

FIGS. 2A-2C depict another embodiment of a collection device 200 that includes a removable sheath 202. As shown in FIG. 2A, the sheath 202 may completely cover the interior components of the collection device 200 and be removable from a handling area 204. For example, the sheath 202 may be removable from the handling area 204 by a temporary attachment mechanism. In some embodiments, the temporary attachment mechanism can be a twist mechanism (e.g., screw fitting), or a pressure mechanism (e.g., snap fitting). In general, the connection made by any temporary attachment mechanism used in a collection device 200 can be sufficient to prevent the flow of liquid.

In some embodiments, the removable sheath 202 can have an interior diameter that is approximately the same as the outer diameter of the swab 210. In some embodiments, the sheath 202 can have an interior diameter larger than the approximate outer diameter of the swab 210. A solution chamber 206 is depicted within the handling area 204 and can be unsealed through any mechanism described herein. In such embodiments, the solution released by the unsealed solution chamber 206 can be manually mixed (e.g., shaken) by the user within the sheath 202.

In some embodiments, the collection device 200 can include one or more structures that are operable to perform particular functions upon user actuation. For example, an actuator can include, but is not limited to, a depressible button, a lever, a switch, twistable mechanism, or a removable gate.

FIG. 2B shows the collection device 200 with the sheath 202 removed from the handling area 204, thereby exposing the shaft 208 and the swab 210. The specimen can be collected with the exposed swab 210 and the sheath 202 reaffixed to the handling area to protect the integrity of the collected specimen.

FIG. 2C depicts another embodiment of the collection device 200 of FIG. 2A. The sheath 202 is depicted as having an optional element 220 at the distal end of the sheath 202. The optional element 220 can house the sealed solution chamber 206 prior to sample collection. The sealed chamber 206 can be unsealed using any suitable mechanism as described herein.

The attachment of the handling element 204 and sheath 202 can be designed in such a manner to have a partially attached arrangement and a fully attached arrangement. For example, the partially attached arrangement can allow the handling element 204 and sheath 202 to be handled by a user while maintaining the sterility of the interior lumen area. After sample collection, the swab 210 can be returned to the sheath 202 and the handling area 204 driven to the fully attached arrangement. The fully attached arrangement can include a means to puncture or unseal the solution container. The fully attached arrangement, for example, can cause the tip of the swab 210 to puncture the solution chamber 206 within the optional module 220 and allow the solution to mix with the sample within the sheath 202.

A second optional element 221 is shown affixed to the proximal end of the handling element 204. The second optional element 221 can be a removable element. The second optional element 221 can be removable (e.g., twist off) after the handling element 204 has been fully attached to the sheath 202 such that the sample/solution mixture can flow from the sheath 202, through the handling element 204, and be directed into a testing strip.

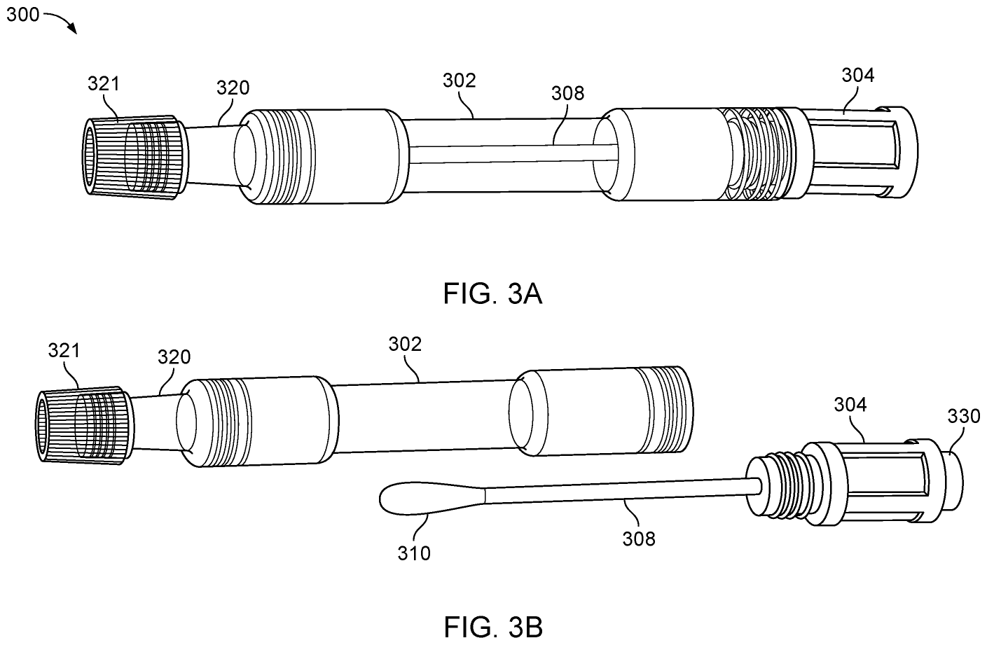

FIGS. 3A-3B depict another embodiment of a collection device 300. As depicted, the sheath 302 of the collection device 300 can include one or more optional elements (e.g., one or more, two or more, three or more). FIG. 3A depicts an embodiment in which the removable sheath 302 includes an optional removable element 320 at the distal end of the sheath 302.

In general, the optional element 320 can be temporarily attached via any removable mechanism disclosed herein. In some embodiments, the optional element 320 can be a functional element (e.g., a needle tip, a suction tip). In some embodiments, the optional element 320 can be an extensible portion of the sheath 302 to allow for varying lengths of shaft 308. In some embodiments, the optional element 320 can include one or more additional elements such as a temporarily attached cap 321 as depicted in FIG. 3A. Alternatively, the optional element 320 can include the solution chamber of the collection device 300. Similarly to FIG. 2C, the optional element 320 can include a partially or fully attached arrangement where the solution chamber can remain sealed until the user operates the optional element 320 to the fully attached arrangement. The solution chamber can then be pierced and the sample mixed with the solution.

FIG. 3B shows the handling area 304 and affixed shaft 308 and swab 310 removed from the sheath 302 and an optional element 330 at the proximal end of the handling area 304. The optional element 330 of FIG. 3B can be an actuator for the unsealing of one or more solution chambers. The optional element 330 can also be a removable element to allow the flow of solution through the handling element 204, as described in FIG. 2C. In some embodiments, a collection device 400 is configured without a sheath.

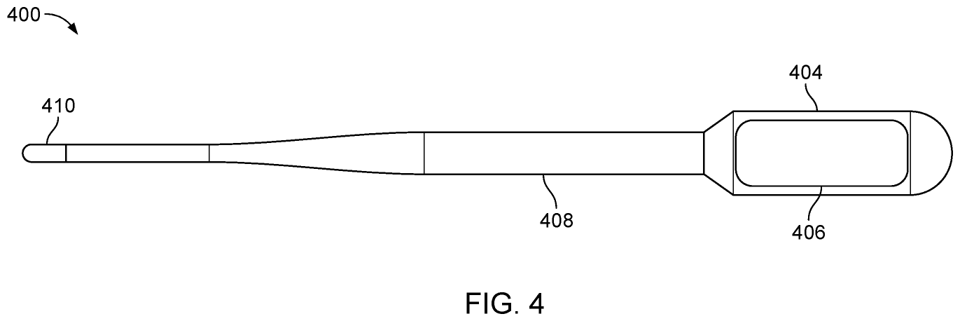

FIG. 4 depicts an exemplary collection device 400 that includes a handling area 404 configured as a bulb, a shaft 408, and a swab 410. In the exemplary embodiment of FIG. 4, the swab 410 is partially enclosed within the distal lumen of the shaft 408. This configuration can allow a specimen to be collected on the partially enclosed swab 410, the contents of the solution chamber 406 to flow through the shaft 408 and saturate the specimen and swab 410 without contamination.

In some embodiments, the swab 410 can be designed to absorb the correct specimen volume and provide a visual indication when the correct specimen volume is collected. For example, when the collection device 400 is used to collect a blood specimen, the absorbent plug may turn progressively red as the blood specimen is wicked into the swab 410. As the specimen is absorbed from the distal tip of the swab 410 to the proximal end enclosed within the shaft 408, the color front can indicate when the correct specimen volume is collected, e.g., when the color front reaches the proximal end of the swab 410.

The exemplary collection device 400 of FIG. 4 depicts the handling area 404 as permanently affixed (e.g., integrated with) the shaft 408. In some embodiments, the handling area 404 may contain one or more solution chambers 406 within the lumen area of the handling area 404, or within the shaft 408.

FIG. 4 further depicts a shaft 408 with a tapered distal end. In general, the shaft 408 of a collection device 400 can be of any profile that facilitates collection of a specimen. For example, the shaft 408 profile can include, but is not limited to, tapered, rounded, beveled, or uniform. The shaft 408 profile can be symmetric about the length of the shaft 408 or the profile can be non-symmetric. The shaft 408 may contain the solution chamber 406.

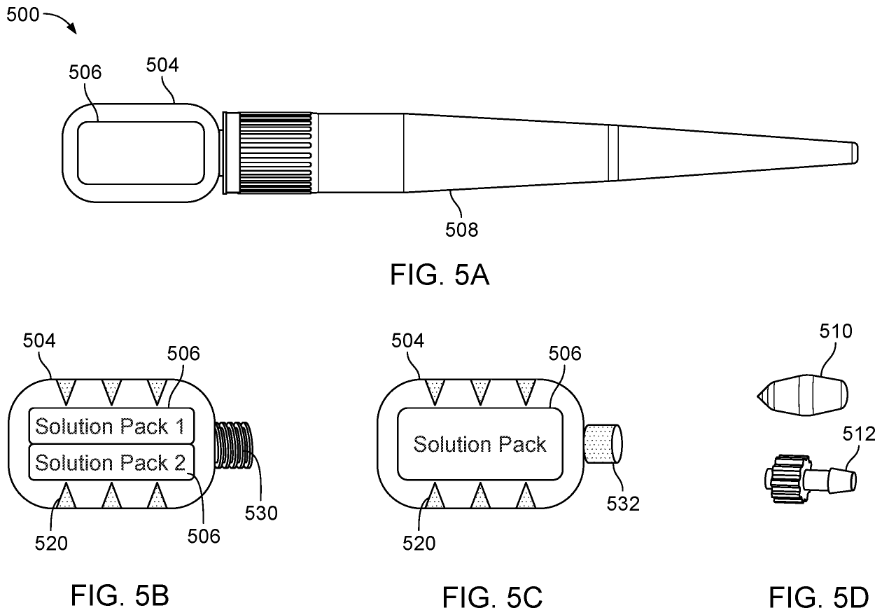

Referring now to FIG. 5A-5C, in some embodiments, the collection device 500 can be composed of one or more interchangeable elements. In some embodiments, the collection device 500 can include a removable handling area 504 element. The handling area 504 element may be removably attached to the shaft 508 via any suitable mechanism known in the field. The removable attachment between one or more elements can create a seal that allows the liquid from one element to flow to another without leakage or contamination. FIG. 5A-C depict exemplary collection device 500 elements. FIG. 5A depicts an exemplary removable handling area 504 element attached to a shaft 508 element. The exemplary modular handling area 504 in FIG. 5A is depicted with a single solution chamber 506.

FIG. 5B depicts an exemplary handling area 504 element with a male screw-type attachment 530. In general, the screw-type attachment 530 can be male-threaded or female-threaded. In some embodiments, the handling area 504 element can attach to an exemplary shaft 508 element or solution chamber 506 element with a female or male screw-type receiving port.

In some embodiments, the handling area 504 element can include a means of unsealing the one or more solution chambers 506 contained within the lumen of the handling area 504 element. FIG. 5B depicts pointed triangular formations 520 affixed to the interior walls of the handling area 504 element (e.g., needles, or teeth). FIG. 5B depicts a handling area 504 element containing two solution chambers 506 though, in general, there can be one or more solution chambers 506 as described above.

FIG. 5C depicts an exemplary handling area 504 element with a solution chamber 506 surrounded by triangular formations 520 and a snap-type temporary attachment port 532. The snap-type attachment port 532 can provide attachment to an exemplary shaft 508 or solution chamber 506 element with a receiving snap-type attachment port. In some embodiments, the action of attaching (e.g., threading, or snapping) a handling area 504 element to an exemplary shaft 508 or solution chamber 506 element can cause the solution chamber 506 element to become unsealed, thereby releasing the contents.

FIG. 5D depicts exemplary interchangeable swab 510 and tip 512 elements. In some embodiments, the interchangeable swab 510 or tip 512 element can attach to a shaft 508 element through any means of temporary attachment described herein. In some embodiments, the swab 510 or tip 512 element can be covered by a temporary or retractable sheath, as described herein. A swab 510 or tip 512 element can be composed of any material disclosed herein. The interchangeable swab 510 or tip 512 elements can be used to facilitate different modes of sample collection (e.g., contact absorption, or sample extraction).

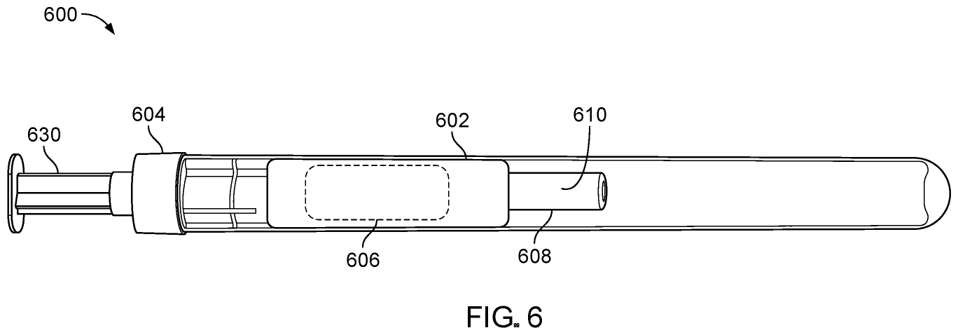

FIG. 6 depicts an exemplary collection device 600 with a handling area 604, a sheath 602, and a shaft 608. The swab 610 is depicted as fully enclosed within the shaft 608. FIG. 6 further depicts an actuator 630 (e.g., a plunger) at the proximal end of the collection device 600. In some embodiments, the swab 610 and the actuator 630 can be connected via a rod (not shown). In some embodiments, the connecting rod can be composed of a rigid or semi-rigid material (e.g., plastic, glass, metal, or wood).

In some embodiments, the actuator 630 can unseal the one or more solution chamber 606 contained in the handling area 602 and extend the swab 610 from the shaft 608 via the connecting rod. In some embodiments, the swab 610 extension can be directly controlled by the actuator 630. In some embodiments, the swab 610 can be partially or fully extendible by the actuator 630. In some embodiments, the swab 610 can be extendable with one operation of the actuator 630 and retractable with a second.

FIG. 7A-7C depict exemplary swab 710 element profiles for use with a collection device. FIG. 7A depicts an exemplary rectangular swab 710 element. FIG. 7B depicts an exemplary tapered cylindrical swab 710 element. FIG. 7C depicts a tapered conical swab 710 element with a hemispherical tip.

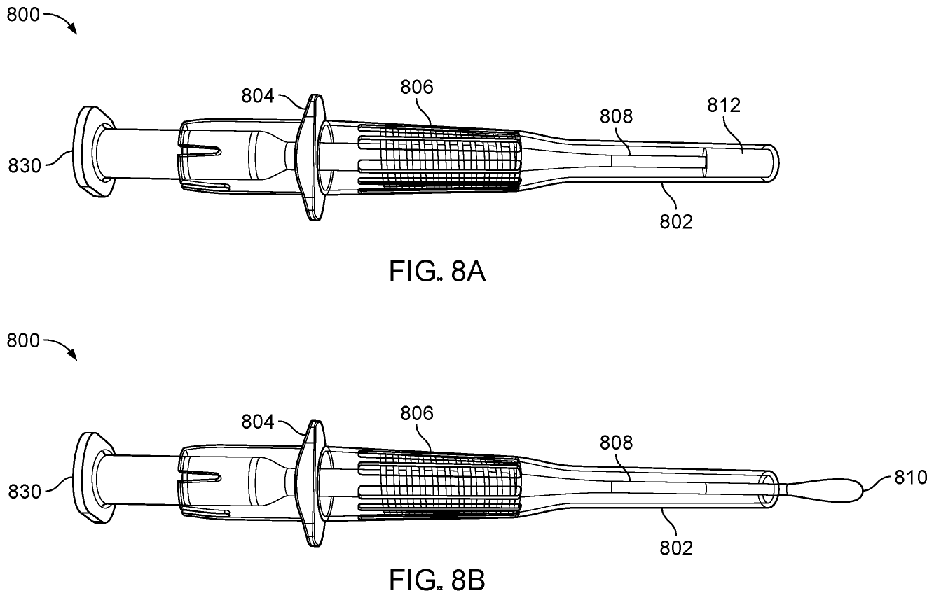

FIG. 8A depicts an embodiment of an exemplary collection device 800 with an actuator and tip element. In general, collection device 800 includes a sheath 802 that defines a hollow shaft housing interior components of the collection device 800.

The interior components of the collection device 800 can include a handling area 804, a sealed solution chamber 806, and a shaft 808. In some implementations, the handling area 804 includes one or more buffer buttons (not shown) that screw or snap into place and are configured to be filled separately as well as easily assembled (e.g., like collection tips). The buffer button(s) can have a capacity of 0.3 or 0.4 mL and can be modified to support up to three times that amount to allow for flexibility in use and use cases.

FIG. 8A further depicts an actuator 830 (e.g., a plunger, a button) at the proximal end of the collection device 800. In some embodiments, a swab at the distal end of the collection device 800, as depicted in FIG. 8B, and the actuator 830 can be connected via a rod (e.g., the shaft 808). In some implementations, the connecting rod is composed of a rigid or semi-rigid material (e.g., plastic, glass, metal, or wood). The collection device 800 further include a tip element 812 at the distal end of device 800. A diameter of the tip element 812 can be smaller than a diameter of a central portion of the body of device 800. For example, the flair of the device 800 can be reduced by 20% at the tip element 812. In some implementations, the actuator 830 can be depressed like a button to puncture a seal that releases solution in the solution chamber 806. Pressing down on the actuator 830 can also push down a rubber gasket within sheath 802, positioned behind the solution chamber 806, thereby gently pushing or plunging the solution down the shaft 802.

FIG. 8B depicts another image of collection device 800 with the actuator 830 engaged, thereby extending swab 810 extending from the sheath 802. In some implementations, actuator 830, when depressed (actuated), causes unsealing of the one or more solution chambers 806 within handling area 804. Depressing the actuator 830 also moves swab 810 via the shaft 808 from an retracted position within sheath 802 to an extended position outside of sheath 802. In some implementations, the swab 810 extension can be directly controlled by the actuator 830. In some implementations, the swab 810 can be partially or fully extendible by the actuator 830. In some implementations, the swab 810 can be extendable with one operation of the actuator 830 and retractable with a second.

The collection device 800 can be adjustable and/or fixed. When the collection device 800 is in an adjustable mode, the tip element 812 can be opened or removed to protrude outside the sheath 802. The tip element 812 can be a collection tip that screws or snaps onto the shaft 808 and/or the sheath 802. Since the tip element 812 can be easily removed and attached, swab tips 810 can be interchangeable. Moreover, different use cases can be supported (e.g., finger stick, nasal swab, etc.). In some examples, tip element 812 can be opened or removed from the device 800 so that swab 810 extends at least partially out of the collection device 800. As another example, and as depicted in FIG. 8B, once the tip element 812 is removed, the actuator 830 can be selected/actuated (e.g., depressed, rotated, and/or turned) to cause swab 810 to extend from the sheath 802 into an open position. Thus, the user can open, collect a biological specimen, and close the collection device 800 with a simple action applied to the actuator 830

As an example, when in adjustable mode, the actuator 830 can be twisted, which causes the tip element 812 and/or the swab 810 to extend from the shaft 808 and/or the sheath 802 into an open position. The swab 810 can be fully revealed once in the open position. In other examples, the swab 810 can be partially revealed in the open position. The swab 810 can remain stable in the open position such that reasonable downward pressure does not push the swab 810 back inside the sheath 802. The user can then open, collect a biological specimen, and close the collection device 800. When the collection device is in a closed position, the swab 810 and/or the tip element 812 can be inside the shaft 808 and/or the sheath 802. For example, the tip element 812 can be a leur-lock cap (e.g., screw or cap) that can be used to maintain the swab 810 sterile inside the sheath 802. In some implementations, the tip element 812 can also allow a buffer to be released into the shaft 808 to move with a collected biological specimen.

When the collection device 800 is in a fixed mode, the tip element 812 and/or the swab 810 can be locked in a fixed position and preventing from moving the fixed position (e.g., a latch can be provided in device 800 that locks the shaft 808 in place). In some implementations, the tip element 812 can be made of a sponge material. The tip element 812 can be set to protrude ˜20% of its total current length. The tip element 812 can also be set so that little or minimal sponge or material of the tip element 812 is left inside the shaft 808 and/or the sheath 802 (e.g., when the actuator 830 is fully depressed).

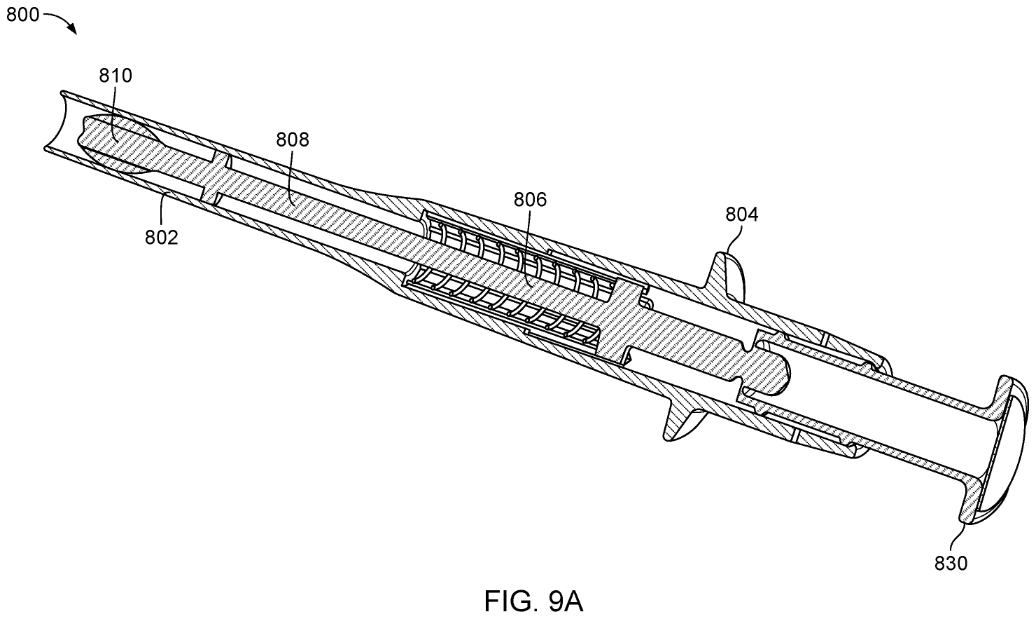

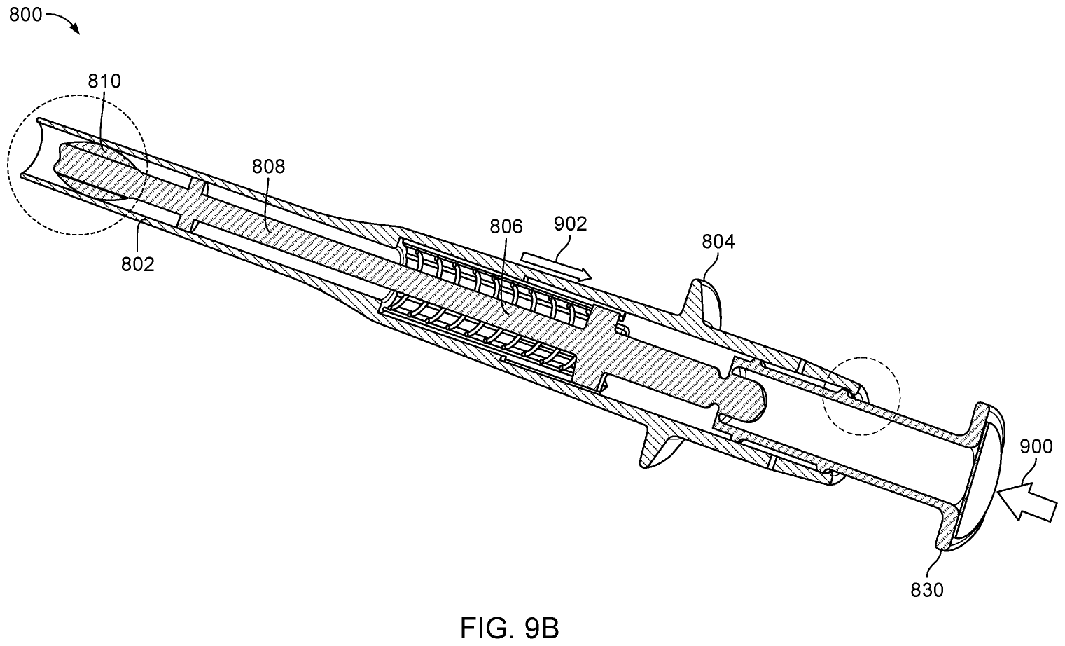

FIG. 9A is a cross-sectional view of exemplary collection device 800 with the actuator 830 and swab 810 retracted within the sheath 802. Generally, when the collection device 800 is in a closed position and/or before the collection device 800 is used, the swab 810, as attached to the shaft 808, can be retracted inside the sheath 802. Therefore, the swab 810 can be kept sterile from exposure to external elements.

FIG. 9B is a cross-sectional view of exemplary collection device 800 with the swab 810 retracted within the sheath 802 when the actuator 830 is engaged. In general, when the actuator 830 of the collection device 800 is pressed down in a direction 900 along the longitudinal axis of the collection device 800, the shaft 802 moves through solution chamber 806, thereby causing solution within the solution chamber 806 to move down towards the swab 810. Pressing upon the actuator 830 also causes pressure in the reverse direction 902, which punctures a seal in the solution chamber 806. Once the seal in the solution chamber 806 is punctured, a solution inside the solution chamber 806 can flow down towards the swab 810.

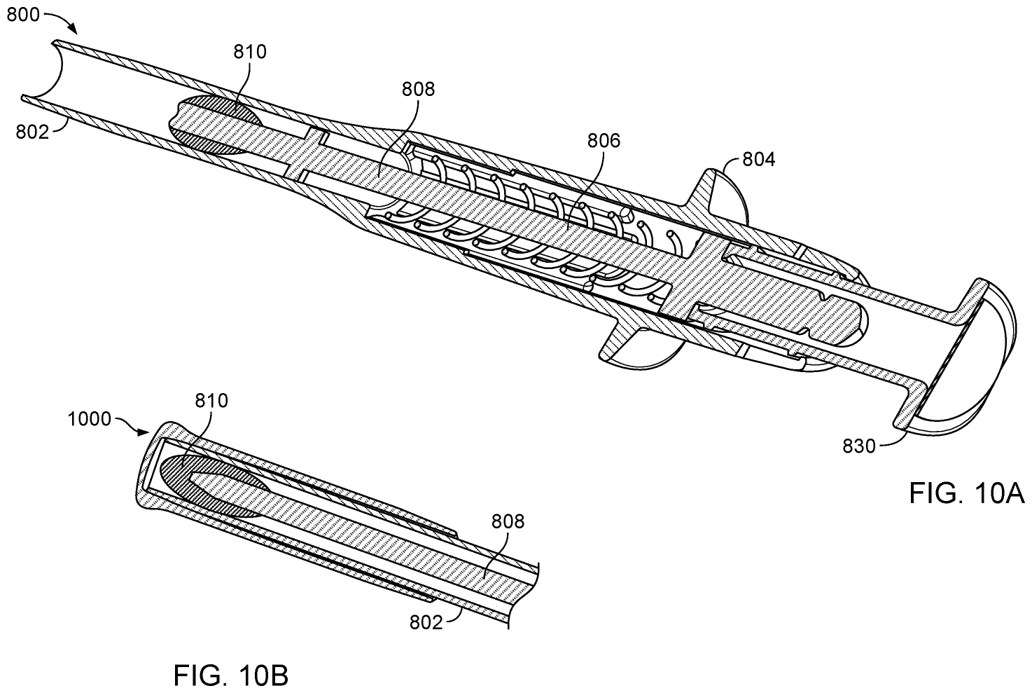

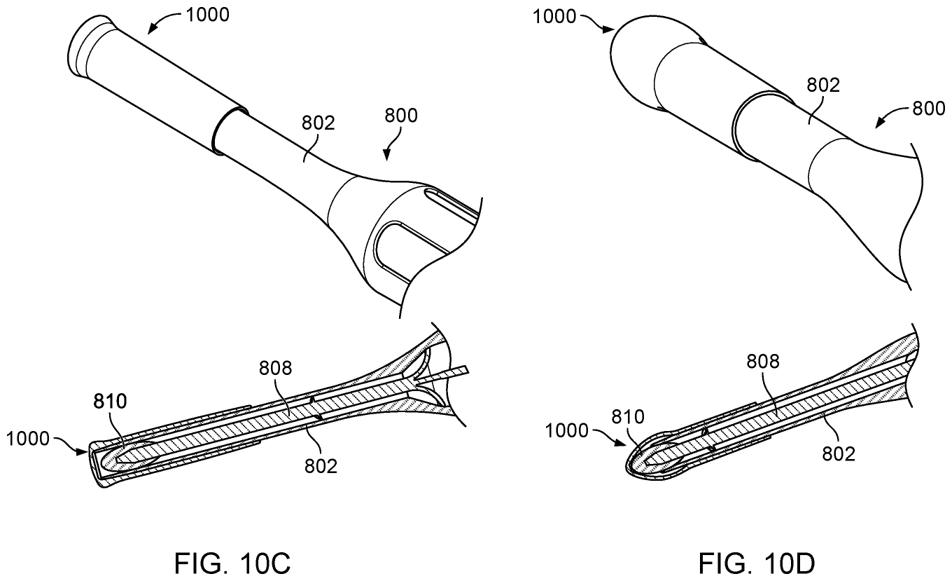

FIG. 10A is a cross-sectional view of an exemplary collection device 800 in which the swab 810 is retracted within the sheath 802 when the actuator 830 is activated. Generally, once a biological specimen has been collected using swab 810 in the extended position, the user can twist, rotate, pull up on, or otherwise disengage the actuator 830 to cause swab 810 to retract back within the sheath 802. This movement can also cause a solution in the solution chamber 806 to release into the sheath 802 and soak the swab 810.

FIG. 10B is a cross-sectional view of an exemplary collection device 800 having a cap 1000 configured to seal the swab within sheath 802. For example, a cap fixture 1000 can be attached to the distal end of the collection device 800 once the user has finished collecting a biological specimen. The cap 1000 can have a small notch or screw mechanism that allows for the cap 1000 to be screwed or snapped into place in a way that makes the collection device 800 waterproof. As depicted, the cap 1000 can screw or snap into place around a tip of the collection device 800 and over sides of the sheath 802. This configuration can be advantageous to provide for collecting specimens and preserving them while the collection device 800 with the collected specimen is mailed to a lab for processing.

In some implementations, cap 1000 includes a transport medium instead of a bugger. As a result, the cap 1000 can be placed back on the collection device 800 and a top of the cap 1000 can be twisted to release the transport medium. Once the top of the cap 1000 is twisted and the transport medium is released, the collection device 800 can be transported or mailed to the lab for processing.

FIG. 10C depicts an exemplary rectangular cap 1000 configured to attach to the sheath 802 of an exemplary collection device 800. The cap 1000 can close around a tip of the collection device 800. The cap 1000 can extend over a portion of the sheath 802, which can be advantageous to provide a barrier against water intrusion and to preserve the collected specimen located within the device 800. When the cap 1000 is snapped into position on the collection device 800, an interior side of the cap 1000 can be flush or pressed flat against the tip of the sheath 802, where the swab 810 would normally extend out of. This configuration can be advantageous to preserve the collected specimen on the swab 810.

FIG. 10D depicts yet another embodiment of cap 1000 configured to attach to the sheath 802 of an exemplary collection device 800. As depicted, the cap 1000 has a curved shape around its distal tip (e.g., as opposed to the flat/rectangular shape shown in FIG. 10C) that mirrors a shape of the swab 810. The cap 1000 can be configured to close around the tip of the collection device 800. The cap 1000 can extend over a portion of the sheath 802, which can be advantageous to provide a barrier against water intrusion and to preserve the collected specimen located within the device 800. When the cap 1000 is snapped into position on the collection device 800, an interior side of the cap 1000 fits closely around the swab 810. This configuration can be advantageous to preserve the collected specimen on the swab 810. Moreover, as depicted, this configuration is especially advantageous where the swab 810 does not fully retract back within the sheath 802 after the biological specimen is collected. At least a portion of the swab 810 can remain extended out from the sheath 802 and the curved cap 1000 can still be placed around the swab 810. The cap 1000 can be shaped to accommodate for one or more different types of swabs or other collection tips or elements.

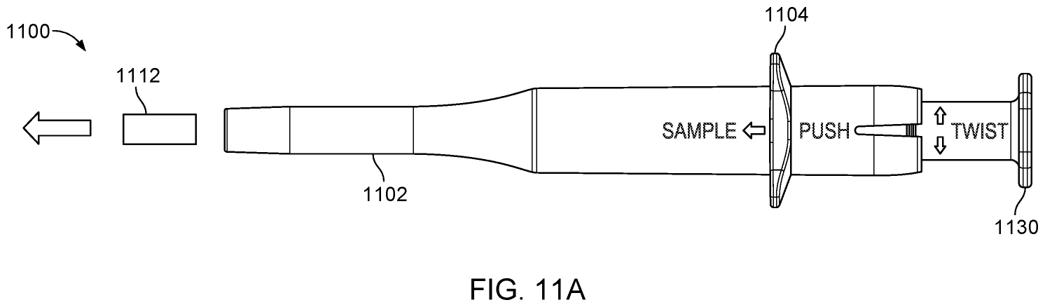

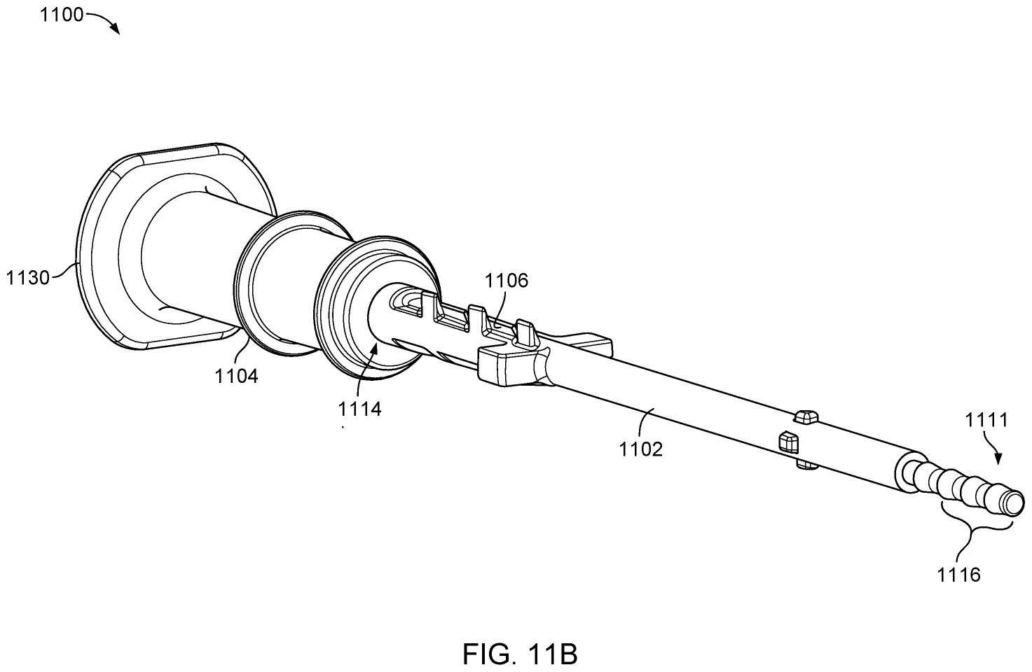

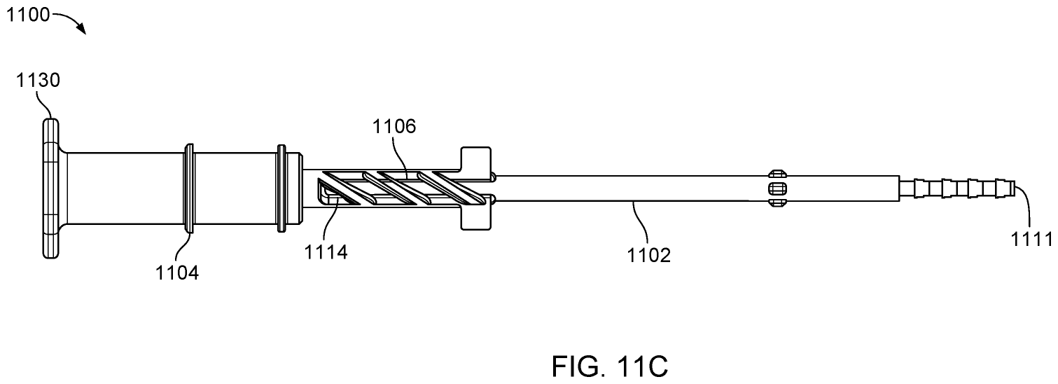

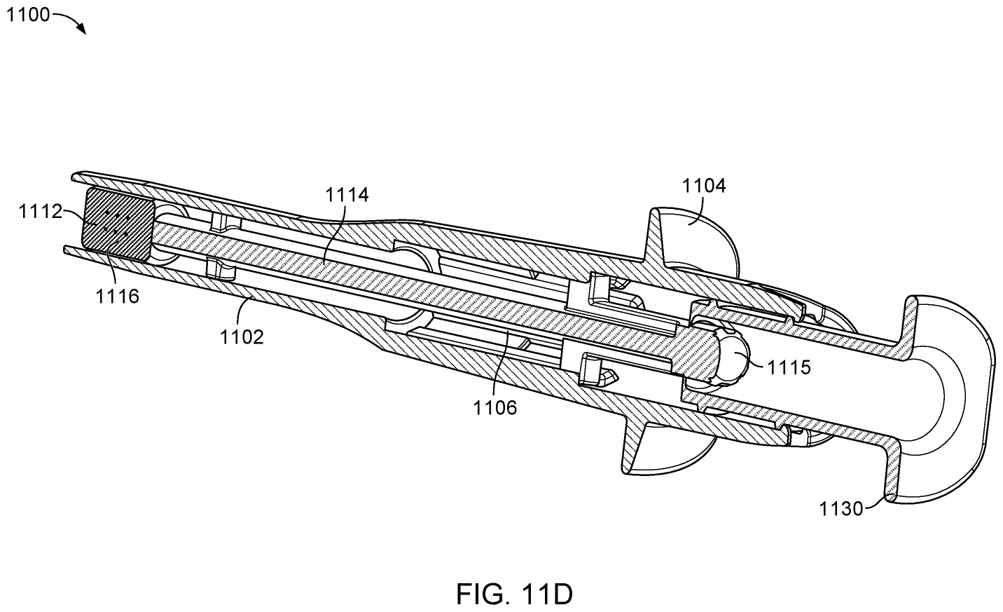

FIG. 11A depicts an exemplary collection device 1100 having an actuator 1130 and a tip element 1112. In particular, the collection device 1100 in this embodiment can include an actuator 1130, a handling area 1104, a sheath 1102, and a tip element 1112. The tip element 1112 can be assembled with the collection device 1100 from a front end of the device 1100. When the actuator 1130 is engaged (e.g., depressed/pushed down and/or twisted down), an ampul barrier optionally can be displaced. Moreover, a protruding length of the tip element 1112 can be enlarged to 11.7 to 12.0 mm, as one example. Moreover, the length of the tip element 1112 can be extended to greater than or equal to 13 mm. In some implementations, the tip element 1112 can be a non-moving element for collecting specimen as well as a moving element. This dual use can be advantageous for fingerstick use cases (e.g., non-moving) and nasal use cases (e.g., moving). For example, the tip element 1112 can be non-retractable after the ampul barrier is broken. A diameter of the tip element 1112 can also change to incorporate both moving and non-moving use cases. Therefore, a universal tip element 1112 can be used with the collection device 1100 for different use cases.

FIG. 11B depicts another view of the exemplary collection device 1100. The collection device 1100 includes an interior shaft 1114 that a swab or other absorbent tip, such as the tip element 1112, is affixed to. The interior shaft 1114 can be hollow so that when a seal of a solution chamber 1106 is broken or otherwise opened, solution flows inside the interior shaft 1114 and down to the swab or the tip element 1112. A tip 1111 of the interior shaft 1114 can have a screw or snap mechanism configured to connect single purpose or interchangeable absorbent tip(s), such as swabs and the absorbent tip element 1112. The tip 1111 of the interior shaft 1114 can also have one or more holes 1116 to which the absorbent tip connects. In some implementations, a lower portion or sides of the interior shaft 1114 includes the one or more holes 1116. With this connection point, when the solution releases within the interior shaft 1114, the solution can be distributed through the absorbent tip component so as to mix more effectively with a collected specimen. This configuration can provide for more effective flow of the solution through and around the device 1100.

FIG. 11C depicts a side view of the exemplary collection device 1100 having the actuator and the tip element. The collection device 1100 of FIG. 11C is described in reference to FIGS. 11A-D.

FIG. 11D is a cross-sectional view of the exemplary collection device 1100 having the actuator 1130 and tip element 1112 with the tip element retracted within sheath 1102. Pushing the actuator 1130 (or turning or rotating a top section of the device 1100 and/or the actuator 1130) can cause seal 1115 to break. This seal 1115 can be part of the solution chamber 1106. Breaking or otherwise opening the seal 1115 can create a plunging force that causes solution to flow down the interior shaft 1114 toward the swab and/or the tip element 1112 (e.g., absorbent tip element). A gasket plunger can force the solution to flow down the shaft 1114 when punctured, thereby pushing the gasket down behind the solution chamber 1106.

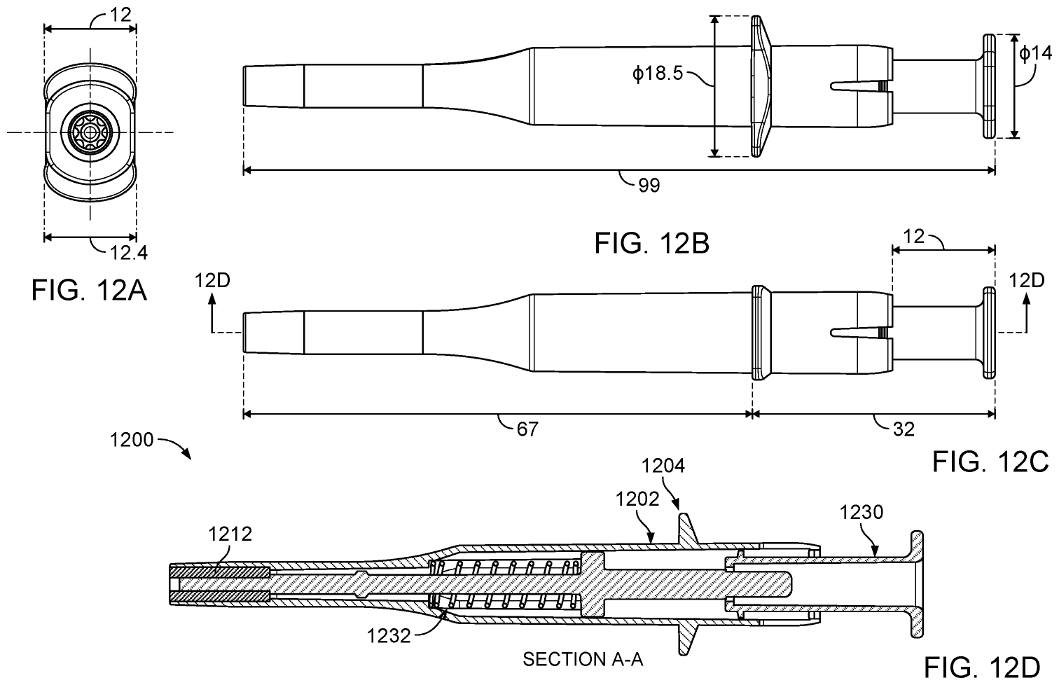

FIGS. 12A-D respectively depict a front view, first side view, second side view, and cross-sectional view of an exemplary collection device 1200. The device 1200 includes a body or sheath 1202, a circular protrusion forming a handling area 1204, a tip element 1212, actuator (e.g., plunger) 1230, and a compressed/biased spring 1232.

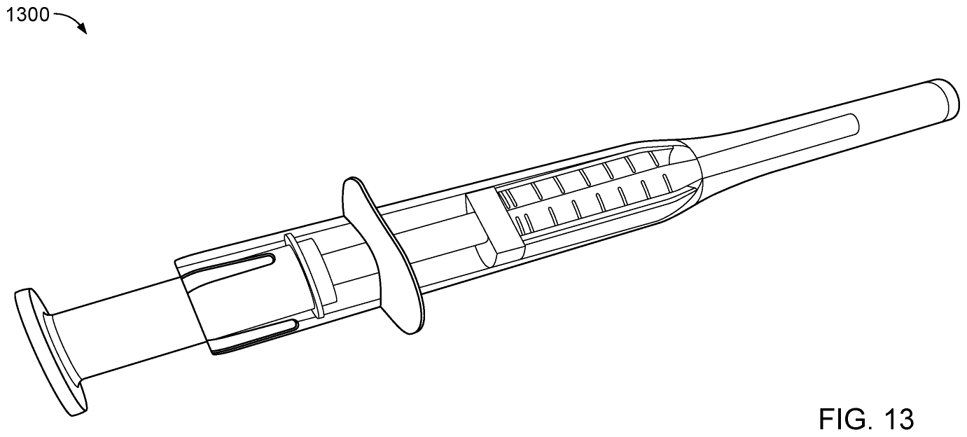

FIG. 13 is a perspective view of an exemplary collection device 1300.

Although various examples have been described in detail above, other modifications are possible. Accordingly, other implementations are within the scope of the following claims.

1. A device for collecting a material specimen, comprising:
a sheath; and
an inner assembly, comprising:
a handling element;
one or more fluid chamber elements; and
a swab element;
wherein:
the handling element, the one or more fluid chamber elements, and the swab element are connected and arranged along a first axis; and
the inner assembly is operable to slide within the sheath between (i) a first position in which the swab element is in a non-retracted position outside of the sheath while at least a portion of the inner assembly remains within the sheath, and (ii) a second position in which the swab element is in a retracted position within the sheath.

2. The device of claim 1, wherein the sheath includes a second axis, and the first axis of the inner assembly is configured to slide coaxially along the second axis of the sheath.

3. The device of claim 1, wherein the handling element is located at a proximal end of the inner assembly, the swab element is located at a distal end of the inner assembly, and the one or more fluid chamber elements are located intermediately between the proximal end of the inner assembly and the distal end of the inner assembly.

4. The device of claim 3, wherein the inner assembly further includes a shaft that extends along the first axis between the one or more fluid chamber elements and the swab element.

5. The device of claim 1, wherein the handling element comprises a compressible bulb configured to expel a pressurized gas to the one or more fluid chamber elements when the bulb is compressed, thereby causing a fluid within the one or more fluid chamber elements to be expressed toward the swab element.

6. The device of claim 5, wherein the inner assembly is configured such that expulsion of the pressurized gas from the bulb when compressed causes a seal in the one or more fluid chamber elements to be broken to permit flow of the fluid.

7. The device of claim 1, wherein the material specimen comprises saliva, urine, blood, or serum of a person.

8. The device of claim 1, wherein the device is configured to collect material specimens of multiple types.

9. The device of claim 1, wherein actuation of the inner assembly breaks a seal in the one or more fluid chamber elements to permit a flow of a fluid from the one or more fluid chamber elements.

10. The device of claim 1, wherein at least one of the handling element or the swab element is a modular component that is removably connected to the inner assembly.

11. A device for collecting a material specimen, comprising:
an assembly, comprising:
a handling element;
one or more fluid chamber elements; and
a swab element;
wherein:
the handling element, the one or more fluid chamber elements, and the swab element are connected and arranged along a first axis.

12. A device for collecting a material specimen, comprising:
a sheath;
a plunger configured at a distal end of the sheath; and
an inner assembly, comprising:
a handling element;
one or more fluid chamber elements;
a shaft; and
a swab element;
wherein:
the handling element, the one or more fluid chamber elements, the shaft, and the swab element are connected and arranged along a first axis;
the inner assembly is operable to slide within the sheath between (i) a first position in which the swab element is in a retracted position within the sheath, and (ii) a second position in which the swab element is in a non-retracted position outside of the sheath while at least a portion of the inner assembly remains within the sheath; and
actuating the plunger causes the inner assembly to slide within the sheath between the first position and the second position.

13. The device of claim 12, wherein the plunger comprises a button.

14. The device of claim 12, wherein actuating the plunger comprises twisting the plunger around a center axis.

15. The device of claim 12, wherein actuating the plunger comprises depressing the plunger into the sheath.

16. The device of claim 12, wherein:
the one or more fluid chamber elements retain a fluid; and
actuating the plunger causes a seal of the one or more fluid chamber elements to break, thereby causing the solution to flow from the one or more fluid chamber elements to the swab element while the swab element is in the retracted position within the sheath.

17. The device of claim 12, wherein the swab element is detachable from the inner assembly.

18. The device of claim 12, wherein a second swab element can be connected to the shaft of the inner assembly.

19. The device of claim 12, wherein depressing the plunger causes the inner assembly to slide within the sheath from the first position to the second position.

20. The device of claim 12, wherein pulling up on the plunger causes the inner assembly to slide within the sheath from the second position to the first position.
